(19) United States
(12) Patent Application Publication
(10) Pub. No.: US 2007/0196832 A1
(43) Pub. Date: Aug. 23, 2007

(54) Methods for mutation detection

(76) Inventors: J. William Efcavitch, San Carlos, CA (US); Marie Sutherlin Causey, Cambridge, MA (US)

Correspondence Address: GOODWIN PROCTER LLP;PATENT ADMINISTRATOR, EXCHANGE PLACE, BOSTON, MA, 02109-2881, US

(21) Appl. No.: 11/360,859

(22) Filed: Feb. 22, 2006

(51) Int. Cl. C12Q 1/68; C12P 19/34

(52) U.S. Cl. 435/6; 435/91.2

(57) ABSTRACT

The invention relates to methods for detecting a mutation in a nucleic acid. Methods of the invention are useful for detecting and identifying mutations that are indicative of disease or the predisposition for disease.

### FIELD OF THE INVENTION

The invention relates to methods for detecting a mutation in a target nucleic acid.

### BACKGROUND

Many diseases are associated with genomic instability. As such, instability markers have been proposed as diagnostic tools. For example, mutations are considered valuable markers for a variety of diseases, and have formed the basis for screening assays. The detection of specific mutations can be a basis for molecular screening assays for the early stages of certain types of cancer. For example, mutations in the BRCA genes have been proposed as markers for breast cancer, and mutations in the p53 cell cycle regulator gene have been associated with the development of numerous types of cancers.

Early mutation detection allows early disease diagnosis, and thus also provides an avenue for intervention prior to the presentation of disease symptoms that often occurs after metastasis when a cure is less readily attainable. However, the detection of genetic mutations or other alterations is difficult, or impossible, in certain sample types. For example, the difficulty of isolating nucleic acid from complex, heterogeneous samples makes identification of early-stage mutations difficult. Furthermore, conventional sequencing technology has limitations in cost, speed, and sensitivity. For example, current sequencing techniques typically involve either an in vitro or an in situ amplification step that requires that the target nucleic acids are present in sufficient copy numbers to achieve the required signal.

Single molecule techniques eliminate the need for costly and often problematic procedures such as cloning and PCR amplification. More particularly, single molecule sequencing methods reduce costs and avoid potential biases that result from bulk techniques such as sequences that amplify poorly. In addition, single molecule techniques require less starting material that conventional sequencing. However, current single molecule sequencing techniques are inaccurate and have limited read-length that makes difficult the ability to detect the presence or absence of mutations in a target nucleic acid.

Therefore, there is a need in the art for efficient methods for determining the presence or absence of certain genetic mutations or other alterations in a target nucleic acid.

### SUMMARY OF THE INVENTION

The present invention provides significant advantages over conventional extension assays which are generally dependent on amplification (e.g., PCR amplification) of each mutation locus on the assumption that the single base extension primer hybridizes correctly to the amplicon and interrogates only the mutation in question. The highly multiplexed nature of the present invention offers advantages over traditional single base extension mutation genotyping. The invention involves several nucleotides of sequence information flanking the site suspected of containing a mutation (e.g., a SNP) which allows greater specificity than is provided by simply hybridizing a single based extension primer alone. The high rate of false positives which can be produced by simply scoring incorporation of a single base extension reaction is avoided by the incorporation of a short stretch of sequence information flanking the incorporated primer(s).

The multiplexed single molecule sequencing readout enables the entire mutation (e.g., a SNP) interrogation reaction to be performed in one reaction tube and then simultaneously decoded on a surface. The density of the single molecule sequencing surface readout means that the highly multiplexed SNP interrogations can be hybridized to a relatively small area. Accordingly, a small surface area can allow for high magnitude of individual mutation interrogations as described herein.

The present invention provides methods for detecting a mutation in target nucleic acids indicative of genomic instability. For example, methods of mutation detection are useful to detect and/or to identify mutations or other alterations associated with diseases, such as cancer and other pathological genetic conditions, disorders or syndromes. Such mutations include nucleotide insertions, deletions, rearrangements, transitions, translations, tranversions, polymorphisms, and substitutions. More specifically, mutations can include single nucleotide polymorphisms (SNP's). The present invention can be used to identify the presence or absence of mutations. Generally, mutations can include any change in the target nucleic acid, such as a loss of heterozygosity or other indicia of genomic instability.

Generally, methods for detecting a mutation in a target nucleic acid include exposing a target nucleic acid template suspected to contain a mutation to a primer that is capable of hybridizing to a known region proximate to the suspected mutation. The primer is extended and one or more complementary nucleotides are hybridized through the site suspected to contain the mutation. The presence or absence of a mutation is determined by analyzing the nucleotides that are incorporated into the primer.

In one aspect, methods for detecting a mutation in a target nucleic acid include a first exposing step, namely, exposing a target nucleic acid template suspected to contain a mutation to a primer. The primer is capable of hybridizing to a known region of the target nucleic acid proximate to the mutation to form a target/primer duplex. A second exposing step includes exposing the target nucleic acid downstream of the known region to one or more labeled nucleotides in the presence of a polymerase, incorporating one or more labeled nucleotide complementary to the target into the primer downstream of the duplex, and identifying the incorporated labeled nucleotide. The identifying step can include exposing the incorporated labeled nucleotide to light that excites a fluorescently labeled nucleotide. The second exposing step, the incorporating step, and the identifying step are repeated one or more times. The sequence of the target nucleic acid is determined by compiling the detected nucleotides, thereby determining the complimentary sequence of the target nucleic acid. Repeating the second exposing step, the incorporating step, and the identifying steps enables determination of a sequence of the target nucleic acid based upon the order of the incorporation of the labeled nucleotide(s). The nucleic acid sequence detects the presence or absence of the suspected mutation in the target nucleic acid. Where the determined sequence of the target is complementary to the wild type the absence of mutation is confirmed. In instances where the determined sequence of the target nucleic acid does not correspond to the wild type a mutation is detected. The mutation can be determined by comparing the nucleic acid sequence to the expected wild type sequence. Accordingly, a determined sequence that differs from the wild type is a positive assay for a mutation in the target nucleic acid.

Methods also can include an optional step of digesting the target. Optionally, the target nucleic acid is sheared prior to exposing the target nucleic acid template suspected to contain a mutation to a primer capable of hybridizing to a known region proximate to the mutation. In one embodiment, the target nucleic acid is first sheared and second is digested. For example, the target nucleic acid is sheared to a size ranging from about 2 kb to about 1 kb, preferably about 1.5 kb. The target nucleic acid can be digested by, for example, exposure to DNase I digestion to a size ranging from about 250 bp to about 50 bp, preferably about 150 bp. The target nucleic acid can be individually optically detectable. In one embodiment, the incorporated labeled nucleotide is individually optically resolvable.

In another aspect, methods for detecting a mutation in a target nucleic acid include exposing a target nucleic acid template suspected to contain a mutation to a primer. The primer is capable of hybridizing to a known region proximate to the mutation. Extending the primer through a site suspected to contain the mutation in the presence of at least one nucleotide and a polymerase. Optionally, each of the at least one nucleotides are unlabeled. The extended primer is detached from the target and a complement is hybridized to the detached extended primer to form an individually-optically detectable duplex. The complement is exposed to at least one labeled nucleotide and the incorporated labeled nucleotide is identified. Methods can include the optional step of exposing the complement to a plurality of chain terminating nucleotides. In one embodiment, the identifying step includes exposing the incorporated labeled nucleotide to light that excites the fluorescently labeled nucleotide.

As discussed herein, the target nucleic acid can be individually optically detectable. In one embodiment, the incorporated labeled nucleotide is individually optically resolvable. The exposing and identifying steps are repeated one or more times. The exposing and identifying steps enable determination of a sequence of the target nucleic acid based upon the order of incorporation of the complementary labeled nucleotide. By determining the target nucleic acid sequence, one can determine whether a mutation is present or absent. For example, where the determined nucleic acid sequence is complementary to the wild type the absence of mutation in the target nucleic acid is confirmed. In instances where the determined nucleic acid sequence does not correspond to the wild type a mutation is detected, the mutation can be identified by comparing the determined nucleic acid sequence to the wild type. Accordingly, a determined nucleic acid sequence that differs from the wild type is a positive assay for a mutation in the target.

In accordance with invention, the primer can be upstream of the mutation, for example, in one embodiment, the 5′ end of a hybridized primer is between about 1 base and about 20 bases from the site suspected to contain a mutation.

In single molecule sequencing, the target nucleic acid molecule/primer duplex is immobilized on a surface such that nucleotides added to the immobilized primer are individually optically resolvable. The primer, template and/or nucleotide analogs can be detectably labeled such that the position of the duplex is individually optically resolvable. Optionally, the duplex can be immobilized on a surface such that the duplex is individually optically resolvable prior to the addition of any nucleotides.

The primer can be attached to a solid support, thereby immobilizing the hybridized target nucleic acid molecule, or the target nucleic acid can be attached to the solid support thereby immobilizing the hybridized primer. The primer and the target can be hybridized to each other prior to or after attachment of either the template or the primer to the solid support. For example, the target nucleic acid can be bound to a surface or support, such as glass. The glass support can have an epoxide coating. In addition, the multiplexed single molecule sequencing enables the entire mutation interrogation reaction to be performed in one reaction tube and then simultaneously decoded on a surface. The density of the single molecule sequencing surface readout means that the highly multiplexed mutation interrogations can be hybridized to a small area. For example the area can be less than 10 mm². Accordingly, a surface having dimensions 3.5 cm×3.5 cm can be modified such that about 100 individual 500,000 mutation interrogation reactions, as described herein, could be applied to the surface and readout simultaneously by the single molecule imaging system.

The target can be attached directly to the support via an amine linkage or a linker pair. Suitable linker pairs can be selected from biotin/avidin, antigen/antibody, and receptor/ligand. In one embodiment, each of a plurality of targets is bound to a support. The target can be exposed to a plurality of different nucleotide species, each having a different detectable label. Any detectable label can be used in practice of the method. The labeled nucleotide can be optically-detectable such as, for example, a fluorescent label. Examples of appropriate fluorescent labels include cyanine, rhodamine, fluorescien, coumarin, BODIPY, alexa, conjugated multi-dyes, or any combination of these. Other detectable labels appropriate for methods of the invention are known to those skilled in the art.

After the target nucleic acid is exposed to a primer that hybridizes to a region proximate to a suspected mutation, the primer/target nucleic acid duplex is extended by exposure to one or more nucleotide and a polymerase under conditions suitable to extend the primer in a template dependent manner. For example, in one embodiment, a Klenow fragment with reduced exonuclease activity is used to extend the primer in a template-dependent manner. Generally the primer/target nucleic acid duplex allows template dependent nucleotide polymerization. The primer is extended by one or more bases. The polymerase can be selected from, for example, Klenow, Nine degrees north, Vent, Taq, Tgo, sequenase, or any combination of these. The nucleotide can include a removable blocking group attached to the 3′ hydroxyl.

The hybridization melting temperature of each primer can be about the same. In one embodiment, the primers are between about 1 bp and about 30 bp long. Optionally, each primer is the same length, i.e., composed of the same number of nucleotide base pairs. The primers can be labeled by, for example, an optically detectable label. Suitable labels can include fluorescent labels. In a multiplex reaction primers can be differentially labeled to aid in mutation detection and/or determination.

While the method is exemplified herein with fluorescent labels, the method is not so limited and can be practiced using nucleotides labeled with any detectable label, including chemiluminescent labels, luminescent labels, phosphorescent labels, fluorescence polarization labels, and charge labels.

The methods of mutation detection include detecting the presence or absence of a mutation at a genetic locus of the target nucleic acid. Any mutation associated with a disease can be detected according to the present invention. Such mutations associated with a disease include, for example, CARD15, SERCA2b, GSTM-1, NAT2, NOD2, ABCA3, K-RAS, p53, APC, DCC, or BAT26. The mutation can be associated with cancer, such as lung cancer, esophageal cancer, prostate cancer, breast cancer, pancreatic cancer, stomach cancer, liver cancer, colon cancer, or lymphoma. The mutation also can be associated with other diseases or disorders, such as Alzheimer's, Parkinson's, and Crohn's disease, for example. In accordance with the methods of mutation detection, the presence or the absence of mutation can be detected and, upon detecting the presence of a mutation, the type of mutation (e.g., a K—RAS mutation) can be determined.

A detailed description of the certain embodiments of the method is provided below. Other embodiments of the invention are apparent upon review of the detailed description and the drawings that follows.

### BRIEF DESCRIPTION OF THE DRAWINGS

FIG. 1 shows a schematic method of mutation detection that includes hybridizing a primer to a known region proximal to a suspected mutation and incorporating labeled nucleotides.

FIGS. 2A-2B show a schematic method of mutation detection that includes hybridizing a primer to known region proximal to a suspected mutation and extending the primer/target nucleic acid through the suspected mutation.

### DETAILED DESCRIPTION OF THE INVENTION

The invention relates generally to methods for detecting the presence or absence of a mutation in a target nucleic acid. The detection methods are particularly suited to single molecule sequencing. The method of mutation detection avoids read-length and accuracy limitations in single molecule sequencing that limit the ability to detect the presence or absence of mutations in a target nucleic acid via single molecule sequencing.

Single molecule sequencing has the inherent advantage of working directly from genomic DNA, thereby eliminating the need for DNA amplification (PCR). In addition to greatly simplifying the overall sample preparation process, this abolishes the introduction of amplification errors and bias, and ultimately reduces cost. By eliminating amplification, nucleic acid molecules can be closely packed on the substrate, thereby providing the largest amount of sequence information from a given surface area. The entire human genome can be represented on a single, compact, glass substrate, for example. Imaging a substrate densely packed with individually-resolvable, single molecules of nucleic acid provides the largest amount of sequence information per image, thus per unit time, enabling the sequencing of entire genomes in a day as opposed to years. As such, these advantages translate directly into reductions in cost both in terms of sample preparation and sequencing chemistry. In addition, reagent use is orders of magnitude lower than alternative amplification based technologies for the equivalent amount of data.

Specifically, the methods employ a primer that is capable of hybridizing to a known region proximate to a suspected mutation in a target nucleic acid template. After primer incorporation, the presence or absence of mutation is detected by nucleotides that incorporate through the region of suspected mutation. Hybridizing the primer to the known region proximate to the suspected mutation limits the number of single molecules required to incorporate into the template to enable mutation detection via sequence identification. Thus, incorporation error and the impact of read length limitations are reduced. Optionally, the methods of mutation detection can be employed in a highly parallel multiplexed assay. The time required to detect the presence or absence of mutation is reduced in this method versus where single molecule sequencing is used alone. The target and/or the incorporated nucleotides can be individually optically resolvable.

### Nucleic Acid Sequencing

The invention includes methods for detecting a mutation in a target nucleic acid. The methods for mutation detection are particularly suited to single molecule sequencing techniques. Such techniques are described for example in U.S. patent application Ser. Nos. 10/831,214 filed April 2004; Ser. No. 10/852,028 filed May 24, 2004; Ser. No. 10/866,388 filed Jun. 10, 2005; Ser. No. 10/099,459 filed Mar. 12, 2002; and U.S. Published Application 2003/013880 published Jul. 24, 2003, the teachings of which are incorporated herein in their entireties.

In general, methods for mutation detection include exposing an individually optically resolvable target nucleic acid template (also referred to herein as template nucleic acid or template) to a primer that is complimentary to at least a portion of the target nucleic acid, under conditions suitable for hybridizing the primer to the target nucleic acid proximate to a mutation. The primer is capable of hybridizing to a known region proximate to the mutation, forming a target nucleic acid/primer duplex.

Target nucleic acids include deoxyribonucleic acid (DNA) and/or ribonucleic acid (RNA). Target nucleic acid molecules can be obtained from any cellular material, obtained from an animal, plant, bacterium, virus, fungus, or any other cellular organism. Target nucleic acids may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the method of mutation detection. Nucleic acid molecules may also be isolated from cultured cells, such as a primary cell culture or a cell line. The cells from which target nucleic acids are obtained can be infected with a virus or other intracellular pathogen.

A sample can also be total RNA extracted from a biological specimen, a cDNA library, or genomic DNA. Nucleic acid typically is fragmented to produce suitable fragments for analysis. In one embodiment, nucleic acid from a biological sample is fragmented by sonication. Test samples can be obtained as described in U.S. Patent Application 2002/0190663 A1, published Oct. 9, 2003, the teachings of which are incorporated herein in their entirety. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, target nucleic acid molecules can be from about 5 bp to about 20 kb. Nucleic acid molecules may be single-stranded, double-stranded, or double-stranded with single-stranded regions (for example, stem- and loop-structures).

The method of mutation detection relies upon the use of primers. Each primer is a single-stranded nucleic acid. According to the method, a primer hybridizes to its complementary region on the target nucleic acid. More particularly, the primers' complementary region on the target nucleic acid is a known region that is proximal to a suspected mutation.

In practicing the present method, the target nucleic acid is incubated with one or more primers. In one embodiment, the primer is bound to a support such as a solid phase or semi-solid phase matrix. The length of individual primers may be from about 4 to about 100 nucleotides. In a preferred embodiment, individual primers are from about 8 to about 30 nucleotides in length. Primers comprising RNA, DNA, and/or Peptide Nucleic Acid (PNA) may be employed to hybridize to the target nucleic acid. The primers may be synthesized chemically by methods that are standard in the art, e.g., using commercially-available automated synthesizers.

One or more of the primers may be labeled. For example, fluorochromes (such as FITC or rhodamine), enzymes (such as alkaline phosphatase), biotin, or other well-known labeling compounds may be attached directly or indirectly to the primer. Alternatively, the primer may be radioactively labeled or conjugated to other commonly used labels or reporter molecules. Further, the primers can be marked with a molecular weight modifying entity (MWME) that uniquely identifies each of the primers.

The primer hybridization reaction can be performed under conditions in which primers having different nucleic acid sequences hybridize to their complementary DNA with equivalent strength. This is achieved by: 1) employing primers of equivalent length; and 2) including in the hybridization mixture appropriate concentrations of one or more agents that eliminate the disparity in melting temperatures (Tₘ) among primers of identical length but different guanosine+cytosine (G+C) content. Thus, under these conditions, the hybridization melting temperatures (Tₘ) of each member of the plurality of single-stranded nucleic acids is approximately equivalent. Agents that may be used for this purpose include quaternary ammonium compounds such as tetramethylammonium chloride (TMAC).

TMAC reduces hydrogen-bonding energy between G−C pairs. At the same time, TMAC increases the thermal stability of hydrogen bonds between A-T pairs. Those opposing influences reduce the difference in normal bond strength between the triple-hydrogen bonded G−C based pair and the double-hydrogen bonded A-T pair. TMAC also increases the slope of the melting curve for each primer. Together, those effects allow the stringency of hybridization to be increased to the point that single-base differences can be resolved, and non-specific hybridization minimized. See, e.g., Wood et al., Proc. Natl. Acad. Sci., U.S.A. 82:1585, (1985), incorporated by reference herein. Any agent that exhibits those properties can be employed in practicing the present method. Such agents are easily identified by determining melting curves for different test primers in the presence and absence of increasing concentrations of the agent. This can be achieved by attaching a target nucleic acid to a solid matrix such as a nylon filter, individually hybridizing radiolabeled primers of identical lengths but different G+C content to the filter, washing the filter at increasing temperatures, and measuring the relative amount of radiolabeled primer bound to the filter at each temperature. Any agent that, when present in the hybridization and washing steps described above, results in approximately superimposable and steep melting curves for the different primers may be used.

In practicing the present method of mutation detection, the target nucleic acid and primers are incubated for sufficient time and under appropriate conditions to maximize specific hybridization and minimize non-specific hybridization. The conditions to be considered include the concentration of each primer, the temperature of hybridization, the salt concentration, and the presence or absence of unrelated nucleic acid.

In one embodiment, each of the primers comprises an equal number of nucleotides. The primer sequences are designed to hybridize to a known region adjacent a suspected mutation in a target nucleic acid. Optionally, the optimal concentration for each primer can be determined by test hybridizations in which the signal-to-noise ratio (i.e., specific versus non-specific binding) of each primer is measured at increasing concentrations of labeled probes.

The temperature for hybridization can be optimized for the length of the primers being used. This can be determined empirically, using the melting curve determination procedure described above. It will be understood by skilled practitioners that hybridization condition determination of optimal time, temperature, primer concentration, salt type, and salt concentration should be done in concert.

According to the method of mutation detection, primers hybridize only to their complementary region on the target nucleic acid. A primer complementary region is a known region proximal to a suspected mutation. After primer hybridization, the target nucleic acid will remain single-stranded about the locus at which a mutation is suspected. An exemplary mutation includes a single nucleotide polymorphism. Following hybridization, unbound primers are, if necessary, removed by washing under conditions that preserve perfectly matched target nucleic acid:primer hybridization products. Washing conditions such as temperature, time of washing, salt types and salt concentrations are determined empirically as described above.

The methods of mutation detection can avoid known polymorphisms being detected as a potential mutation. For example, where one or more polymorphisms are associated with a region of the target nucleic acid, multiple primers, each designed to hybridize to one of the polymorphic variants can be provided. A primer complimentary to a polymorphic variant on the target will hybridize to the region of the polymorphism. Thus, according to the method, primers can be designed to block the known polymorphic variants including known polymorphisms that are proximal to a suspected mutation. Thus, providing primers complementary to each polymorphic variant ensures that the polymorphic region is blocked by a primer and single-stranded regions suspected to contain a mutation that are adjacent the complementary primer can, according to the method, indicate the presence or absence of a mutation other then an associated polymorphic variant on the target nucleic acid.

In one embodiment, the target nucleic acid/primer duplex is individually optically resolvable in order to facilitate single molecule discrimination. The nucleic acid target, the template, the primer, and/or the target/primer duplex can be bound to a support. The choice of a support for attachment depends upon the detection method employed. Preferred supports for use with the method include supports comprising epoxides or a polyelectrolyte multilayer. Such layers or coatings are preferably deposited on a surface that is amenable to optical detection of the surface chemistry, such as glass or silica. The precise support used in the method of mutation detection is, however, immaterial to the functioning of the method described herein. In one embodiment, the support bound nucleic acid duplex is bound to a glass having, for example, an epoxide coating. The duplex can be attached directly to the support via, for example, an amine linkage or a linker pair. Suitable linker pairs are selected from, for example, biotin/avidin, antigen/antibody, and receptor/ligand. In one embodiment, each of a plurality of targets is bound to a support, i.e., multiple targets are bound to a single coated bead by, for example, an amine linkage at an end of each target nucleic acid that links each target nucleic acid to the single bead.

One or more nucleotides and a polymerase are added to the target nucleic acid/primer duplex under conditions suitable for extending the primer in a template-dependant manner. The primer can be extended by one or more nucleotides.

Nucleotides useful in the method include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine. The incorporated nucleotide is identified by, for example, a label present on the incorporated nucleotide. The nucleotide can have a removable blocking group attached to the nucleotides' 3′ hydroxyl. In one embodiment, the target nucleic acid is exposed to a plurality of different nucleotide species each having a different detectable label. The label can be an optically-detectable label such as, for example, a fluorescent label. Each labeled nucleotide species can include a different label, or they can include the same label. An incorporated labeled nucleotide can be individually optically resolvable. The identifying step can include exposing the incorporated labeled nucleotide to light that excites the fluorescent label.

Any polymerase and/or polymerizing enzyme may be employed. A preferred polymerase is Klenow with reduced exonuclease activity. Nucleic acid polymerases generally useful in the method include DNA polymerases, RNA polymerases, reverse transcriptases, and mutant or altered forms of any of the foregoing. DNA polymerases and their properties are described in detail in, among other places, DNA Replication 2nd edition, Komberg and Baker, W. H. Freeman, New York, N.Y. (1991). Known conventional DNA polymerases useful in the method include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9°Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J Biol. Chem. 256:3112), and archaeal DP1I/DP2 DNA polymerase II (Cann et al., 1998, Proc Natl Acad. Sci. USA 95:14250→5).

Other DNA polymerases include, but are not limited to, ThermoSequenase®, 9°Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof. Reverse transcriptases useful in the method include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit Rev Biochem. 3:289-347(1975)).

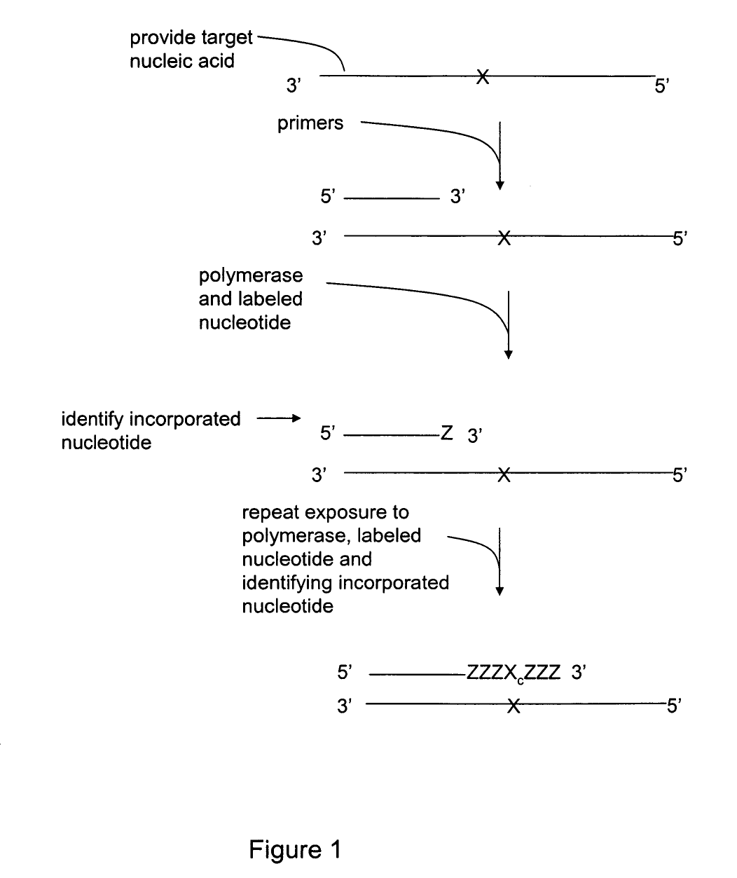

Referring now to FIG. 1, in one embodiment of the method of mutation detection a target nucleic acid suspected to contain a mutation (X) is provided. The target nucleic acid is exposed to a primer capable of hybridizing to a known region proximal to the suspected mutation. Preferably, the primer is exposed to the target under conditions that favor specific hybridization. In one embodiment, multiple primers are provided, however, only one primer hybridizes to the known region proximal to the suspected mutation. A target nucleic acid/primer duplex results and optionally, unbound primers are washed away. The target nucleic acid/primer duplex is exposed to a species of labeled nucleotide in the presence of a polymerase. The labeled nucleotide is incorporated in a template-dependent manner under Watson-Crick base pairing rules. In other words, the nucleotide is incorporated into a primer at a locus at which its complement exists in the template. In an array of duplexes, template-dependent synthesis reactions are driven toward proper incorporation and there is a concomitant reduction in signal from misincorporated bases. Methods of mutation detection include conducting sequencing reactions in the presence of a reaction mixture comprising a polymerase and at least one labeled dNTP corresponding to a first nucleotide species. According to the method, labeled dNTPs that are complementary to an available template nucleotide will result in addition to the read-length. The incorporated labeled nucleotide (Z) is identified by, for example, its label. In cases where labeled nucleotides are incorporated, the label can optionally be bleached and/or cleaved prior to any subsequent synthesis. Exposure of the target/primer duplex to one or more labeled nucleotide in the presence of polymerase is repeated, incorporated nucleotides (ZZZXcZZZ) are identified. The presence or absence of a suspected mutation (X) is detected. In one embodiment, the mutation (X) is detected by incorporation of its complement, Xc. Optionally, the mutation X is determined by comparison of the determined sequence ZZZXcZZZ to the wild type sequence.

The method can involve single molecule sequencing-by-synthesis. Primer/target nucleic acid duplexes are bound to a surface such that one or more duplex is (are) individually optically resolvable. According to the method, a primer/target nucleic acid (template) duplex is exposed to a polymerase and a labeled nucleotide of a first nucleotide species. Optionally, unincorporated labeled nucleotides and/or unincorporated chain elongation inhibitors are washed away. The incorporated labeled nucleotide is identified and, optionally, the optically detectable label is removed from the incorporated nucleotide. In this way, the identity of the nucleotide complementary to a base of the target nucleic acid adjacent the known region proximate to a suspected mutation to which the primer is hybridized is identified (e.g., the base on the target nucleic acid downstream of the primer that hybridized to the known region). The polymerization reaction is serially repeated in the presence of labeled nucleotide that corresponds to each of the four Watson-Crick nucleotide species until a sequence of incorporated nucleotides is compiled from which the sequence of the target nucleic acid through the site suspected to contain the mutation can be determined. Practice of the method results in a majority of duplexes to which the added deoxynucleotide is complementary adding the appropriate (i.e., complementary) nucleotide to the primer.

Optionally, unincorporated nucleotides are removed prior to or after the detecting step. Unincorporated nucleotides can be removed by washing. The target nucleic acid/primer duplex is optionally treated such that the incorporated nucleotide's label is removed, partially removed, degraded and/or a linker that attached the label to the nucleotide is cleaved thereby removing the label. The steps of exposing target nucleic acid/primer duplex to one or more labeled nucleotide and polymerase, detecting incorporated nucleotides, and then treating to (1) remove and/or degrade the label, (2) remove and/or degrade the label and at least a portion of the linker or (3) cleave the linker can be repeated, thereby identifying additional bases in the template nucleic acid, the identified bases can be compiled, thereby determining the sequence of the target nucleic acid. In some embodiments, the label or a remaining linker and label are not removed, for example, in the last round of primer extension.

In one embodiment, in a second exposing step, the target nucleic acid/primer duplex is exposed to one or more labeled nucleotides. The region of the target downstream of the known region to which the primer hybridizes is single stranded. This region of the target downstream of the known region is exposed to the labeled nucleotides. One or more labeled nucleotides complementary to the target nucleic acid are incorporated into the primer such that a labeled nucleotide hybridizes to its single stranded complement on the target nucleic acid.

The incorporated nucleotide is identified by the nucleotide label, for example, the fluorescence of the label. The second exposing step, the incorporating step, and the identifying step are repeated thereby to detect if a suspected mutation is present in the target nucleic acid. The sequence of at least a portion of the target nucleic acid is determined. Comparing the determined nucleic acid sequence versus the wild type sequence enables detection and/or determination of a mutation in the target nucleic acid.

In one embodiment of the method, a target nucleic acid suspected to contain a mutation is provided. The target nucleic acid can be individually-optically detectable and ranges in size from about 5 bp to about 250 bp, preferably to about 150 bp. The target nucleic acid is exposed to a primer capable of hybridizing to a known region proximate to the suspected mutation. In a second exposing step, the target nucleic acid is exposed to one or more labeled nucleotide downstream from the known region in the presence of a polymerase. One or more labeled nucleotides are incorporated into the primer and the incorporated labeled nucleotide is identified. The second exposing step, the incorporating step, and the identifying step are repeated one or more times to detect if the mutation is present in the target nucleic acid.

In one embodiment, the target nucleic acid is prepared by shearing purified genomic DNA with, for example, a Hydroshear device, to from about 2.0 kb to about 1.0 kb, more specifically to about 1.5 kb. Subsequently, the sheared DNA is digested by exposure to, for example, DNase I, to a size ranging from about 5 bp to about 250 bp, preferably to about 150 bp. After inactivation of DNase I, the digested DNA ranges in size from about 5 bp to about 250 bp and is exposed to the method of mutation detection.

In one embodiment the mutation that the target nucleic acid is suspected to contain is a single nucleotide polymorphism (SNP). The prepared DNA is denatured at 95-98° C. for 5 minutes and is then snap cooled in a metal block that has been pre-chilled to 0° C. Once the DNA is denatured the double-stranded DNA separates into individual single strands. It is possible to sequence the suspected SNP from either separated single strand of the DNA duplex, however, one direction may be more useful than another.

In one embodiment, the digested denatured DNA is ready for binding to a SNP specific primer slide. Previously identified and tested SNP specific primers may be used for single molecule sequencing (SMS) SNP detection. Alternatively or in addition, primers can be specifically designed for use in this detection method. Preferably, each primer has similar melting temperatures. In some embodiments, each primer has approximately the same GC-content. Suitable primers are each highly specific to a single SNP. It is also important to consider the SNP sequence when designing SMS SNP primers since the primers, e.g., from about 8 to about 30 bp sequenced tags, must be sufficiently unique to identify the region of interest proximal to any SNP sequence detected therein. Suspected SNP specific primers that hybridize to a given number of known regions proximal to a suspected SNP (e.g., hundreds to thousands of regions proximal to a suspected SNP) are synthetically prepared by a commercial vendor. Optionally, each primer contains a 5′ amine for coupling to epoxide-treated solid surfaces. Slides can be prepared in advance and stored for use as needed.

Hybridization is generally carried out in 3×SSC at elevated temperatures (50-60° C., typically), but more specific hybridization conditions may need to be developed to achieve optimal binding. Suitable specific hybridization conditions employ DTAB and/or formamide in the hybridization buffer to achieve optimal binding of primers with different GC contents.

In one embodiment of the method of mutation detection, SNP specific primers are used to capture sheared target genomic DNA fragments suspected to bear a SNP. The actual genomic DNA is the reverse-complement of the SNP sequence detected. The target nucleic acid suspected to contain a SNP is exposed to a primer capable of hybridizing to a known region of the target proximal to the suspected SNP. In one embodiment, the SNP specific primer hybridizes from about 1 bp to about 20 bp, preferably about 5 bp upstream (5′) of the SNP to be detected.

Subsequent to primer hybridization/capture, the hybridized primers are used to prime DNA synthesis from the gDNA templates. In this way, the target/primer duplex is exposed, in the presence of a polymerase, to one or more labeled nucleotides downstream of the known region proximal to the suspected mutation. One or more labeled nucleotide is incorporated into the primer and the incorporated labeled nucleotide is identified. The steps of exposing target/primer duplex to a labeled nucleotide in the presence of a polymerase, incorporating the labeled nucleotide, and identifying the incorporated labeled nucleotide is repeated to thereby detect if the mutation is present in the target nucleic acid. In this way, enough base pairs to positively identify a unique tag location within the gDNA sequence are added to the primer, e.g., approximately 15 bp to 20 bp are added to the primer. The sequenced tags can be shorter than those used for the whole genome sequencing since the search space will be limited to regions near the regions of GDNA to which SNP specific primers bind. This method effectively reduces the search space complexity and limits the search area to the regions surrounding the site where the primer hybridizes to a region proximate to the suspected mutation, i.e., the search space is proximal to the SNP primer binding sites. The primer and/or the polymerase are selected to determine the direction (e.g., 3′ and/or 5′) that nucleotide addition follows.

In another embodiment of the method, a method for detecting a mutation in a target nucleic acid includes exposing a target nucleic acid template suspected to contain a mutation to a primer. The primer is capable of hybridizing to a known region proximate to the mutation. The primer is extended through a site suspected to contain the mutation in the presence of at least one nucleotide and a polymerase. The extended primer is detached from the target and a complement is hybridized to the detached extended primer to form an individually-optically detectable duplex. Exposing the complement to at least one labeled nucleotide and identifying the incorporated labeled nucleotide. The method can include the optional step of exposing the complement to a plurality of chain terminating nucleotides.

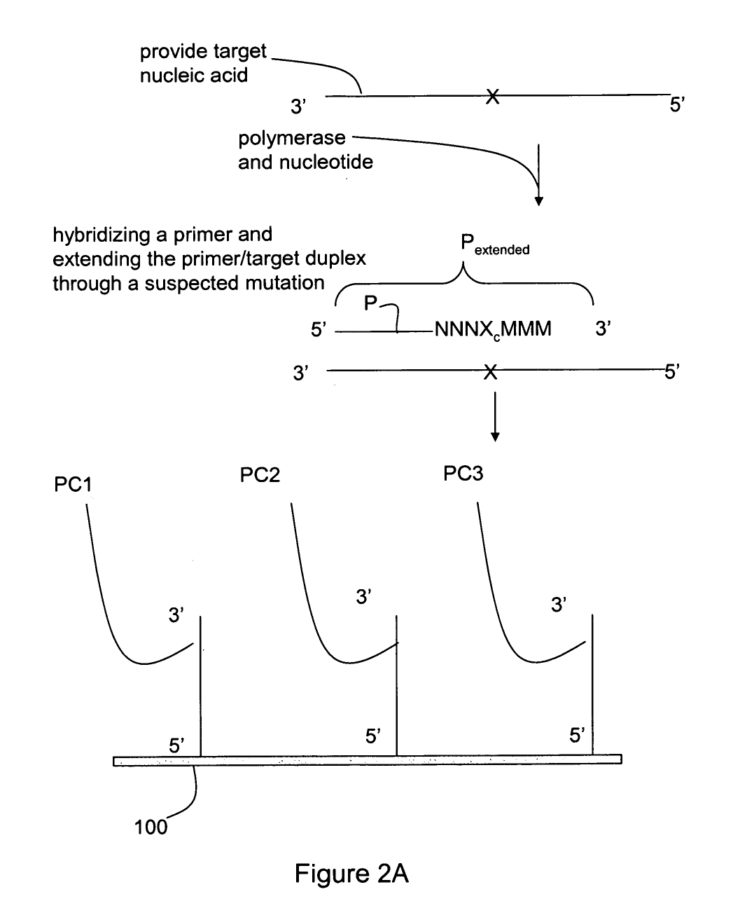

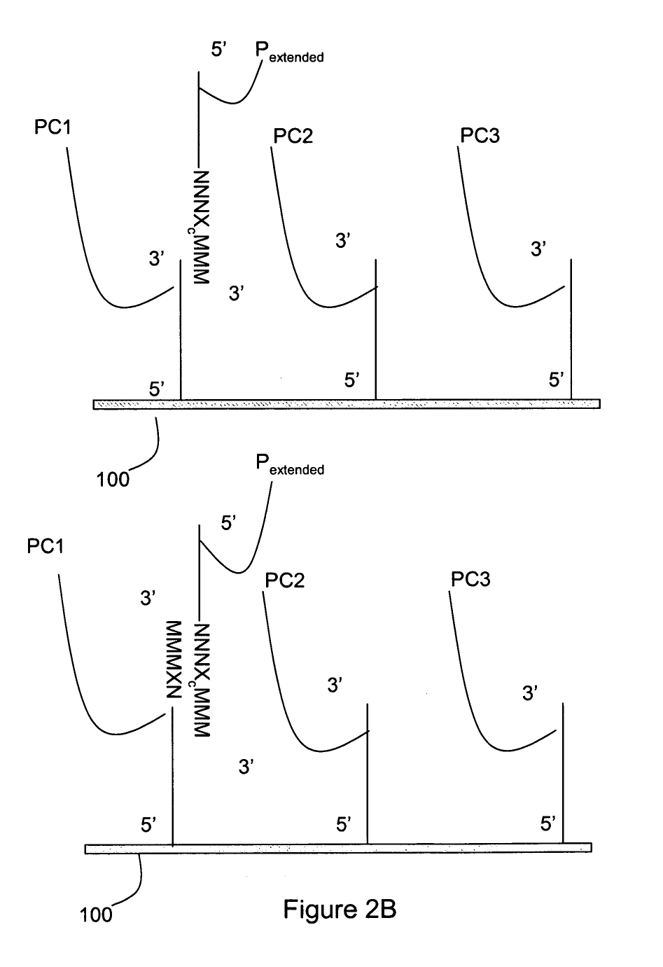

In accordance with this method, highly multiplexed mutation detection can be performed by employing single molecule sequencing as the detector. Referring to FIGS. 2A-2B, this method employs one or more primers (P) that are capable of hybridizing to a known region proximate to a suspected mutation (X) in the target nucleic acid. The primers are designed to have specificity for a region of a target nucleic acid proximal to a mutation, such as for example a SNP to be interrogated according to the method of the mutation detection. Suitable primers include, for example, locus specific oligonucleotide primers (LSOP) that hybridize to a target nucleic acid (e.g., a genomic DNA) in a site specific manner. In one embodiment, the primer is designed to hybridize to a region of the target nucleic acid proximal to a site suspected to contain a mutation. For example, the primer hybridizes to a known region no less than one base from the site suspected to contain a mutation (e.g., the primer is about 1 bp to about 20 bp upstream (5′) of a suspected SNP). Suitable primers are of sufficient length (e.g., sufficient number of base pairs in lengths) to have a specific base sequence in the target nucleic acid being interrogated and/or the target nucleic acid species genome (e.g., the primers have a specific base sequence found in the human genome). The target nucleic acid can be exposed to any number of primers, e.g., from about 1 to about 500,000 different primers.

In one embodiment, the primers are hybridized to the target nucleic acid being interrogated in a one tube reaction. In another embodiment, primers are exposed to the target nucleic acid via a multiplex reaction as are suitable to optimize hybridization. The multiplex reaction can be pooled for the SMS readout step.

Referring still to FIGS. 2A-2B, the primer (P) is hybridized to a known region of a target nucleic acid proximate to a suspected mutation (X), thereby forming a primer/target duplex. After hybridization, the primer/target duplex is exposed to at least one nucleotide in the presence of a polymerase.

In one embodiment, the duplex is exposed to a mixture of a DNA polymerase and a limiting amount of four deoxynucleotide triphosphates that are allowed to extend the primer in a multiplex fashion. In another embodiment, the primer is extended via multiplex reaction for a finite number of nucleotides that incorporate into the primer, e.g., the reaction kinetics enable the primer to be extended by at least 50 bases. Optionally, one or more nucleotides have a removable blocking group attached to the 3′ hydroxyl, which enables the extension reaction to be blocked. Another way of terminating the extension reaction is to include fewer than the four deoxynucleotide triphosphates (e.g., 1-3) thus the polymerase extension is naturally stopped when a base requires one of the absent deoxynucleotide triphosphates and extension stops due to the inability to read over the base missing its complement.

After the duplex extension, the mixture is hybridized to a support 100. The support 100 can be modified with capture primers (PC1, PC2, PC3). The capture primers (e.g., PC1, PC2, PC3) are designed to be complementary to at least a portion of the extended primer (Pextended). More specifically, the capture primers are designed to be complementary to the sequence of the target nucleic acid downstream of the mutation being interrogated (e.g., the capture primer PC1 is designed to be complementary to the sequence MMM that is 3′ of the mutation X in the extended primer, Pextended). The mutation X can be a suspected SNP. Suitable capture primers are designed to have sufficiently high enough melting temperatures (Tₘ's) to survive multiple rounds of single molecule sequencing. There is at least one capture primer for each primer employed in the extension reaction. Thus, where there are 500,000 primers in a multiplex base extension reaction there must be at least 500,000 capture primers attached to the support. Preferably, there are at least 10 capture primers for each primer. The capture primers are oriented such that the 5′ end is attached to the support and the 3′ end is oriented away from the support such that the 3′ end can be employed in the single molecule sequencing reaction. The 3′ end of a captured primer is complementary to the sequence of a target nucleic acid downstream of the mutation. Referring to FIG. 2B, the 3′ end of PC1 is complementary to the sequence of the target nucleic acid complement downstream i.e., 3′ of the mutation X, namely is complementary to the sequence MMM. At least a portion of the captured primer is complementary to the sequence of the target nucleic acid downstream of the mutation, i.e., the portion of the extended primer, Pextended, 3′ of the mutation's complement X_{c}. Preferably, the captured primer is compatible for use with single molecule sequencing (e.g., is stable and has low non specific binding of fluorescently labeled nucleotides).

After hybridization of extended primer Pextended to the captured primer PC1, the multiplexed base extended primers which now encode 1) the sequence of the mutation (e.g., a SNP) being interrogated 2) one or more nucleotide immediately 5′ the mutation being interrogated 3) five or more nucleotides immediately 3′ of the mutation being interrogated, the encoded primers are now subjected to several rounds of single molecule sequencing (SMS). SMS provides sequence information on both sides of the mutation and includes the sequence of the mutation (X) itself. Where the mutation is a SNP, SMS generates a genotype of the SNP. If there are multiple alleles of the SNP more than one sequence will be generated by the SMS process, which enables identification of the SNP alleles present. In this way, the presence or absence of a mutation (X) is detected and where a mutation is detected to be present in the target the type of mutation can be determined by, for example, the nucleic acid sequenced by SMS.

In still another embodiment, a universal primer is employed. This method avoids multiple different types of primers covalently immobilized to a support and instead a universal hybridization support is immobilized to the support. In particular, the sequence determined according to the described method is compared to the wild type to determine the mutation present in the target nucleic acid. According to this embodiment of the method of mutation detection, a primer is hybridized to a known region proximate to suspected mutation in a target nucleic acid. Preferably, the primer hybridizes at least one nucleotide upstream (e.g., 5′) of the suspected mutation, such as, for example, a SNP. The primer is extended by exposing the target/primer duplex to a polymerase in the presence of a nucleotide. Preferably, the primer is extended by at least one nucleotide in the direction downstream (e.g., 3′) of the mutation. The direction of primer extension is controlled by, for example, exposing the mixture to suitable kinetic control. Preferably, the extension is limited such that as few nucleotides as possible extend the primer in the direction downstream (e.g., 3′) of the mutation (e.g., a SNP) being interrogated. The reaction is quickly quenched and an aliquot of dATP and Terminal Deoxynucleotidyl Transferase (TdT) is introduced into the reaction mixture. The TdT is kinetically controlled to allow the incorporation of a suitable number of a single type of nucleotide. In one embodiment at least 5 dA nucleotides are incorporated into the extended primer. In a preferred embodiment, at least 50 dA nucleotides are incorporated into the extended primer. After incorporation of the desired number of dA nucleotides the reaction is terminated by the addition of a large excess of a dideoxy A triphosphate.

After termination of the polymerase extension reaction, the multiplexed mixture is hybridized to a support that is modified to capture primers that are complementary to the multiple incorporated single type of nucleotide e.g., a Poly-A tailed extended primers. According to this method, the capture probes feature multiple nucleotides complementary to the incorporated single type of nucleotide at the free end (e.g., the non-captured end). For example, the a capture probe complementary to Poly-A tailed extended primers each feature a Poly-T sequence.

The SMS of a small portion of sequence information both upstream and downstream of the mutation (e.g., the SNP) associated with a target nucleic acid corrects for any cross hybridization of the primer with the target genomic DNA.

The methods for sequencing a nucleic acid template may employ a label and the label preferably is a detectable label. In one embodiment, the label is an optically-detectable label such as a fluorescent label. The label can be selected from detectable labels including cyanine, rhodamine, fluorescien, coumarin, BODIPY, alexa, conjugated multi-dyes, or any combination of these. However, any appropriate detectable label can be used according to the invention, and are known to those skilled in the art.

### Detection

Any detection method may be used to identify an incorporated nucleotide that is suitable for the type of label employed. Thus, exemplary detection methods include radioactive detection, optical absorbance detection, e.g., UV-visible absorbance detection, optical emission detection, e.g., fluorescence or chemiluminescence. Single-molecule fluorescence can be made using a conventional microscope equipped with total internal reflection (TIR) illumination. The detectable moiety associated with the extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Aca. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached target nucleic acids.

The present method provides for mutation detection in a target nucleic acid, for example, detection of a mutation in a single nucleotide in a target nucleic acid. For example, the methods for detection of a mutation include, for example, a single nucleotide polymorphism (SNP) in a target nucleic acid molecule. A number of methods are available for this purpose. Methods for visualizing single molecules within nucleic acids labeled with an intercalating dye include, for example, fluorescence microscopy. For example, the fluorescent spectrum and lifetime of a single molecule excited-state can be measured. Standard detectors such as a photomultiplier tube or avalanche photodiode can be used. Full field imaging with a two-stage image intensified COD camera also can be used. Additionally, low noise cooled CCD can also be used to detect single fluorescent molecules.

The detection system for the signal may depend upon the labeling moiety used, which can be defined by the chemistry available. For optical signals, a combination of an optical fiber or charged couple device (CCD) can be used in the detection step. In those circumstances where the substrate is itself transparent to the radiation used, it is possible to have an incident light beam pass through the substrate with the detector located opposite the substrate from the target nucleic acid. For electromagnetic labeling moieties, various forms of spectroscopy systems can be used. Various physical orientations for the detection system are available and discussion of important design parameters is provided in the art.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present method use TIRF microscopy for two-dimensional imaging. TIRF microscopy uses totally internally reflected excitation light and is well known in the art. See, e g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached target nucleic acid target molecule/primer complex in the presence of a polymerase. Total internal reflectance fluorescence microscopy is then used to visualize the attached target nucleic acid target molecule/primer complex and/or the incorporated nucleotides with single molecule resolution.

Fluorescence resonance energy transfer (FRET) can be used as a detection scheme. FRET in the context of sequencing is described generally in Braslavasky, et al., Proc. Nat'l Acad. Sci., 100: 3960-3964 (2003), incorporated by reference herein. Essentially, in one embodiment, a donor fluorophore is attached to the primer, polymerase, or template. Nucleotides added for incorporation into the primer comprise an acceptor fluorophore that is activated by the donor when the two are in proximity.

Measured signals can be analyzed manually or by appropriate computer methods to tabulate results. The substrates and reaction conditions can include appropriate controls for verifying the integrity of hybridization and extension conditions, and for providing standard curves for quantification, if desired. For example, a control nucleic acid can be added to the sample. The absence of the expected extension product is an indication that there is a defect with the sample or assay components requiring correction.

In one embodiment, the detectable moiety is attached to the pyrophosphate group, and the pyrophosphate group is removed from the nucleotide analog during primer extension. The pyrophosphate containing the detectable moiety can be removed from the template/primer duplexes into a detection all where the presence and/or amount of the detectable label is determined, for example, by excitation at a suitable wavelength and detecting the fluorescence.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

We claim:

1. A method for detecting a mutation in a target nucleic acid, the method comprising the steps of:
(a) exposing an individually-optically detectable target nucleic acid template suspected to contain a mutation to a primer capable of hybridizing to a known region proximate to said mutation to form a target/primer duplex;
(b) exposing said duplex to one or more labeled nucleotides in the presence of a polymerase;
(c) incorporating one or more labeled nucleotide complementary to said target into said primer downstream of the duplex;
(d) identifying the incorporated labeled nucleotide; and
(e) repeating steps (b)-(d), thereby to determine if the mutation is present in said target.

2. The method of claim 1, wherein said primer is upstream of said mutation.

3. The method of claim 1, wherein said target is bound to a support.

4. The method of claim 3, wherein said support is glass.

5. The method of claim 4, wherein said glass has an epoxide coating thereon.

6. The method of claim 5, wherein said target is attached directly via an amine linkage.

7. The method of claim 5, wherein said target is attached via a linker pair.

8. The method of claim 7, wherein said linker pair is selected from biotin/avidin, antigen/antibody, and receptor/ligand.

9. The method of claim 1, the method further comprising the step of:
(f) shearing the target prior to step (a).

10. The method of claim 9, the method further comprising the step of:
(g) digesting the target.

11. The method of claim 1, wherein a 5′ end of a hybridized primer is between about 1 base and about 20 bases from the site suspected to contain a mutation.

12. The method of claim 1, wherein each of a plurality of targets is bound to a support.

13. The method of claim 1, wherein said polymerase is selected from the group consisting of Klenow, Nine degrees north, Vent, Taq, Tgo, sequenase, or any combination thereof.

14. The method of claim 1, wherein said nucleotide further comprises a removable blocking group attached to the 3′ hydroxyl.

15. The method of claim 1, wherein said target is exposed to a plurality of different nucleotide species, each comprising a different detectable label.

16. The method of claim 1, wherein said labeled nucleotide comprises an optically-detectable label.

17. The method of claim 16, wherein said optically-detectable label is a fluorescent label.

18. The method of claim 17, wherein said identifying step comprises exposing the incorporated labeled nucleotide to light that excites said fluorescent label.

19. The method of claim 1, wherein said incorporated labeled nucleotide is individually optically resolvable.

20. A method for detecting a mutation in a target nucleic acid, the method comprising the steps of:
(a) exposing a target nucleic acid template suspected to contain a mutation to a primer capable of hybridizing to a known region proximate to said mutation;
(b) extending said primer, in the presence of at least one nucleotide, through a site suspected to contain said mutation in the presence of a polymerase;
(c) detaching the primer from said target;
(d) hybridizing a complement to the detached primer to form a duplex, wherein said duplex is individually-optically detectable;
(e) exposing said complement to at least one labeled nucleotide;
(f) identifying the incorporated labeled nucleotide; and
(g) repeating steps (e)-(f), thereby to detect if the mutation is present in said target.

21. The method of claim 20, wherein said primer is upstream of said mutation.

22. The method of claim 20, wherein each of the at least one nucleotides are unlabeled.

23. The method of claim 20, wherein the target is bound to a support.

24. The method of claim 23, wherein said support is glass.

25. The method of claim 24, wherein said glass has an epoxide coating thereon.

26. The method of claim 25, wherein said target is attached directly via an amine linkage.

27. The method of claim 25, wherein said target is attached via a linker pair.

28. The method of claim 27, wherein said linker pair is selected from biotin/avidin, antigen/antibody, and receptor/ligand.

29. The method of claim 20, wherein each of a plurality of targets is bound to a support.

30. The method of claim 20, wherein a 5′ end of a hybridized primer is between about 1 base and about 20 bases from the site suspected to contain a mutation.

31. The method of claim 20, the method further comprising the step of:
(h) exposing said complement to a plurality of chain terminating nucleotides.

32. The method of claim 20, wherein said polymerase is selected from the group consisting of Klenow, Nine degrees north, Vent, Taq, Tgo, sequenase, or any combination thereof.

33. The method of claim 20, wherein said nucleotide further comprises a removable blocking group attached to the 3′ hydroxyl.

34. The method of claim 20, wherein said target is exposed to a plurality of different nucleotide species, each comprising a different detectable label.

35. The method of claim 20, wherein said incorporated labeled nucleotide comprises an optically-detectable label.

36. The method of claim 35, wherein said optically-detectable label is a fluorescent label.

37. The method of claim 36, wherein said identifying step comprises exposing the incorporated labeled nucleotide to light that excites said fluorescent label.

38. The method of claim 20, wherein said incorporated labeled nucleotide is individually optically resolvable.
